(19) United States
(12) Patent Application Publication
(10) Pub. No.: US 2005/0287224 A1
(43) Pub. Date: Dec. 29, 2005

(54) Oxygen generating composition

(75) Inventor: Man-Khyun Rho, Seoul (KR)

Correspondence Address: BACON & THOMAS, PLLC, 625 SLATERS LANE, FOURTH FLOOR, ALEXANDRIA, VA, 22314, US

(73) Assignee: J. C. Technologies, Inc., Seoul (KR)

(21) Appl. No.: 10/916,394

(22) Filed: Aug. 12, 2004

(30) Foreign Application Priority Data
Jun. 23, 2004 (KR) 10-2004-0047084

(51) Int. Cl. A61K 33/40

(52) U.S. Cl. 424/615

(57) ABSTRACT

Disclosed herein are oxygen generating compositions. More specifically, the oxygen generating compositions comprise potassium superoxide or sodium peroxide, a material for stabilizing the reactivity and oxidizing power of potassium superoxide or sodium peroxide, and optionally at least one selected from an oxidation catalyst of carbon monoxide, a material for improving the moldability and processability of the composition and a material for increasing initial carbon dioxide absorption rate. The oxygen generating compositions can be utilized in a wide range of applications.

The material for stabilizing the reactivity and oxidizing power of potassium superoxide or sodium peroxide is selected from calcium hydroxide (Ca(OH)₂), aluminum hydroxide (Al(OH)₃), magnesium hydroxide (Mg(OH)₂), barium hydroxide (Ba(OH)₂), calcium carbonate (CaCO₃), talc and clay. The oxidation catalyst of carbon monoxide is selected from copper oxide (CuO), manganese oxide (MnO) and a mixture thereof (hopcalite). The material for improving the moldability and processability of the oxygen generating compositions is selected from glass powder, glass fiber, ceramic fiber, steel wool, bentonite, kaolinite, sodium silicate and potassium silicate. Since the oxygen generating compositions have stabilized reactivity and oxidizing power, they can be used in household goods. In addition, since the oxygen generating compositions have a higher compressive strength than pure potassium superoxide or sodium peroxide, they can be processed into various shapes.

### TECHNICAL FIELD

The present invention relates to oxygen generating compositions comprising a superoxide or peroxide of an alkali metal.

### BACKGROUND ART

Generally, oxygen generating compounds are used to supply oxygen to airplane passengers in case of emergency where in-flight pressure drops, and to closed areas such as submarines where oxygen supply systems are not normally operated. In addition to these applications, oxygen generating compounds are used in personal portable devices, e.g., oxygen supply devices, for firemen and mine workers in case of emergency.

There are a number of oxygen generating compounds, for example, chlorates and perchlorates of alkali metals, including lithium perchlorate (LiClO₄), lithium chlorate (LiClO₃), sodium perchlorate (NaClO₄), sodium chlorate (NaClO₃), potassium perchlorate (KClO₄), potassium chlorate (KClO₃), etc.

These chlorates or perchlorates generate salts and oxygen during decomposition when being heated by electrical or chemical techniques.

In addition, peroxides and superoxides can be used as oxygen generating compounds. Examples of peroxides include sodium peroxide (Na₂O₂), potassium peroxide (K₂O₂), calcium peroxide (CaO₂) and lithium peroxide (Li₂O₂), and examples of superoxides include sodium superoxide (NaO₂) and potassium peroxide (KO₂).

Among the above-mentioned oxygen generating compounds, potassium superoxide and sodium peroxide are used as air revitalization materials because they can fix carbon dioxide present in air and give off oxygen as depicted in Reactions 1 to 4 below:
Na₂O₂+H₂O→2NAOH+1/2O₂ Reaction 1
Na₂O₂+CO₂→Na₂CO₃+1/2O₂ Reaction 2
2KO₂+H₂O→2KOH+3/2O₂ Reaction 3
2KO₂+CO₂→K₂CO₃+3/2O₂ Reaction 4

As is well known in the art, soda lime, which is a mixture of calcium hydroxide (Ca(OH)₂) and sodium hydroxide, and lithium hydroxide, are widely used as air purifiers which are capable of removing carbon dioxide present in air. However, since these air purifiers do not generate oxygen, they are disadvantageous over sodium peroxide and potassium superoxide in terms of air purification efficiency.

Accordingly, sodium peroxide and potassium superoxide having superior oxygen generating ability are advantageously used in self-contained breathing apparatuses, as compared with the use of carbon dioxide absorbers.

For instance, U.S. Pat. No. 4,490,274 discloses an oxygen generating composition comprising sodium peroxide, potassium superoxide, aluminum hydroxide (Al(OH)₃), manganese dioxide (MnO₂) and powdered aluminum. The composition stably generates oxygen even at a low temperature as well as maintains humidity in the generated oxygen at a constant level.

Although the composition disclosed in U.S. Pat. No. 4,490,274 comprises sodium peroxide and potassium superoxide, potassium superoxide is predominantly used as an air revitalization material in other related techniques due to its high stability and oxygen generation efficiency.

However, potassium superoxide evolves an excess of heat during oxygen generation, resulting in fusion of potassium superoxide pellets. Potassium superoxide also has problems that the time taken to initiate oxygen generation is delayed and it has strong oxidizing power and causticity, and hence is unsuitable for practical use.

Thus, numerous techniques, e.g., addition of a small amount of additives, have been proposed towards finding a satisfactory solution to the above problems.

In an effort to solve the fusion problem of potassium superoxide and further to improve the oxygen generation performance, U.S. Pat. No. 4,113,646 discloses the addition of anhydrous calcium sulfate (CaSO₄), silicon dioxide (SiO₂), lithium monoxide (Li₂O), lithium metaborate (LiBO₂) and the like.

U.S. Pat. No. 4,238,464 discloses a composition for reducing the fusion of potassium superoxide, which comprises a salt containing at least one element selected from zirconium, titanium and boron, and potassium superoxide.

In particular, severe fusion of potassium superoxide inside a bed filled with potassium superoxide creates a considerable pressure drop of air passing through the bed. U.S. Pat. No. 4,490,272 discloses the addition of 2˜30 wt % of an alkaline earth metal oxide, such as CaO, in order to remarkably improve the problem of pressure drop resulting from heat fusion.

In an effort to solve delayed oxygen generation at the initial stage of operation, U.S. Pat. No. 5,690,099 describes an apparatus in which a wetted activated charcoal bed is disposed at one side of a potassium superoxide-filled bed.

The problem of delayed oxygen generation at the initial stage of operation can be avoided by the addition of a small amount of a catalyst to potassium superoxide pellets. For example, German Patent No. 320810 proposes the use of manganese dioxide (MnO₂) as a catalyst. Further, U.S. Pat. No. 4,731,197 reports the addition of copper oxychloride to the surface of potassium superoxide pellets in order to solve the problem of delayed oxygen generation and stably maintain oxygen generation rate.

According to the prior arts, many technical difficulties involved in use of potassium superoxide as an air purifier, i.e. the fusion of potassium superoxide due to evolution of large heat during oxygen generation and delayed oxygen generation at the initial stage of operation, can be overcome. However, no technique has been developed that can reduce the strong oxidizing power, causticity and excessive reactivity of potassium superoxide.

Further, such problems of strong oxidizing power, causticity and excessive reactivity are equally present in sodium peroxide, and are obstacles impeding the application of the oxygen generating compounds to household goods.

Even though it is apparent that sodium peroxide and potassium superoxide can be usefully applied to remove indoor air pollutants, such as carbon dioxide, SOₓ and NOₓ gasses, the removal of the dangerous factors, i.e. strong oxidizing power, causticity and excessive reactivity, is required in order to utilize sodium peroxide and potassium superoxide for household goods such as air conditioner filters.

An improvement in the processability of potassium hydroxide and sodium peroxide is also required in addition to the reduction of causticity and reactivity. Potassium superoxide and sodium peroxide are solid inorganic compounds and are very difficult to process into relatively fine and simple shapes such as pellets. This is because there is no particular process except for powder compression.

Moreover, the kind of binders that can be mixed with potassium superoxide and sodium peroxide is very limited because both potassium superoxide and sodium peroxide are potent oxidants. This limitation must be overcome to obtain various shapes of potassium superoxide and sodium peroxide, e.g., flat-plate filters.

In conclusion, there is a need for compositions capable of reducing and stabilizing the causticity and reactivity of potassium superoxide and sodium peroxide and of improving the processability of the compounds for use in daily life.

### DISCLOSURE

### TECHNICAL PROBLEM

Therefore, the present invention has been made in view of the above problems, and it is a first object of the present invention to provide a highly stable oxygen generating composition capable of reducing the oxidizing power and reactivity of potassium superoxide or sodium peroxide.

It is a second object of the present invention to provide an oxygen generating composition capable of absorbing carbon monoxide.

It is a third object of the present invention to provide an oxygen generating composition with improved processability which can be processed into various shapes.

It is a fourth object of the present invention to provide an oxygen generating composition with a very high initial carbon dioxide absorption rate.

### TECHNICAL SOLUTION

These and other objects are achieved by oxygen generating compositions comprising potassium superoxide or sodium peroxide, a material for stabilizing the reactivity and oxidizing power of potassium superoxide or sodium peroxide, and optionally at least one selected from an oxidation catalyst of carbon monoxide, a material for improving the moldability and processability of the compositions and a material for increasing initial carbon dioxide absorption rate.

The material for stabilizing the reactivity and oxidizing power of potassium superoxide or sodium peroxide is at least one compound selected from alkaline earth metal hydroxides and inorganic fillers.

Examples of alkaline earth metal hydroxides include calcium hydroxide (Ca(OH)₂), aluminum hydroxide (Al(OH)₃), magnesium hydroxide (Mg(OH)₂), barium hydroxide (Ba(OH)₂), etc. Examples of inorganic fillers include calcium carbonate (CaCO₃), talc, clay, etc.

The oxidation catalyst of carbon monoxide is at least one compound selected from copper oxide (CuO), manganese oxide (MnO) and a mixture thereof (hopcalite).

The material for improving the moldability and processability of the oxygen generating compositions is at least one species selected from inorganic binders, such as glass powder, glass fiber, ceramic fiber, steel wool, bentonite, kaolinite, sodium silicate and potassium silicate.

The material for increasing initial carbon dioxide absorption rate is at least one base selected from sodium hydroxide, lithium hydroxide and potassium hydroxide.

### ADVANTAGEOUS EFFECTS

The oxygen generating compositions of the present invention have stabilized reactivity and oxidizing power, and are thus sufficiently safe to be used in household goods.

In addition, since the oxygen generating compositions of the present invention comprise a binder, they have a higher compressive strength than conventional potassium superoxide compositions, enabling processing into various shapes.

Furthermore, since the oxygen generating compositions of the present invention comprise an oxidation catalyst of carbon monoxide, they can absorb carbon monoxide present in air at a very high rate as compared with pure potassium superoxide or sodium peroxide.

### DESCRIPTION OF THE DRAWINGS

The above and other objects, features and other advantages of the present invention will be more clearly understood from the following detailed description taken in conjunction with the accompanying drawings, in which:

FIG. 1 is a graph comparing the carbon dioxide absorption rate of oxygen generating compositions comprising potassium superoxide with that of pure potassium superoxide and sodium peroxide, as a function of time;

FIG. 2 is a graph comparing the carbon dioxide absorption rate of oxygen generating compositions comprising sodium peroxide with that of pure sodium peroxide, as a function of time;

FIG. 3 is a graph comparing the carbon monoxide absorption rate of oxygen generating compositions comprising potassium superoxide and hopcalite with that of pure potassium superoxide, as a function of time;

FIG. 4 is a graph comparing the carbon monoxide absorption rate of oxygen generating compositions comprising sodium peroxide and hopcalite with that of pure sodium peroxide, as a function of time; and

FIG. 5 is a graph comparing the carbon dioxide absorption rate of oxygen generating compositions comprising sodium hydroxide with that of pure potassium superoxide, as a function of time.

### BEST MODE

Hereinafter, best modes for carrying out the present invention will be explained in more detail with reference to oxygen generating compositions described in the following examples.

An oxygen generating composition of the present invention comprises 20˜90 wt % of potassium superoxide or sodium peroxide, and 10˜80 wt % of an alkaline earth metal hydroxide or inorganic filler for stabilizing the reactivity and oxidizing power of the potassium superoxide or sodium peroxide. Preferably, the alkaline earth metal hydroxide or inorganic filler is present in an amount of 40˜70 wt %.

Examples of metal hydroxides include calcium hydroxide (Ca(OH)₂), aluminum hydroxide (Al(OH)₃), magnesium hydroxide (Mg(OH)₂), barium hydroxide (Ba(OH)₂) and the like. Examples of inorganic fillers include calcium carbonate (CaCO₃), talc, clay and the like. These metal hydroxides and inorganic fillers may be used alone or in combination.

In the case where the oxygen generating composition of the present invention is intended to oxidize and absorb carbon monoxide, copper oxide (CuO), manganese oxide (MnO) or a mixture thereof (hopcalite) as a catalyst is added in an amount of 0.01˜5 wt % and preferably 1˜3 wt % based on the total weight of the composition.

For better moldability and processability of the oxygen generating composition, a binder is added in an amount of 0.01˜10 wt % and preferably 2˜7 wt %, based on the total weight of the composition. When the composition is used in a powdery form and thus molding and processing are unnecessary, the binder may be omitted from the composition.

Examples of binders that can be used in the present invention include glass powder, glass fiber, ceramic fiber, steel wool, bentonite, kaolinite, sodium silicate and potassium silicate. These binders may be used alone or in combination.

In the case where the composition of the present invention is intended to increase initial carbon dioxide absorption rate, at least one base selected from sodium hydroxide (NaOH), lithium hydroxide (LiOH) and potassium hydroxide (KOH) is added in an amount of 0.01˜10 wt % to the oxygen generating composition.

The use of the material for stabilizing reactivity and oxidizing power in the composition of the present invention is indispensable, and the use of the other additives, i.e. the oxidation catalyst of carbon monoxide, the binder and the base for increasing initial carbon dioxide absorption rate is optional depending on the intended application.

All materials used in the present invention were of chemically pure (CP) grades, and were dried in a desiccator under nitrogen atmosphere for 48 hours before use. The respective components were mixed in a glove box under nitrogen atmosphere as homogeneously as possible. The mixture was processed using a die and a press at a pressure of 10 tons to form cylindrical pellets.

In the following examples, the reactivity and carbon monoxide removal performance of the oxygen generating compositions, and the strength of the pellets were tested.

### EXAMPLE 1

Pellets having a diameter of 1.0 cm and a height of 1.0 cm were fabricated from the oxygen generating compositions indicated below using a pelletizer. At this time, the fabrication was carried out in a dry atmosphere in order to minimize the influence of moisture.

| SAMPLE A-1: | | |
|---|---|---|
| Potassium superoxide (KO₂): | 30.00 | wt % |
| Calcium hydroxide (Ca(OH)₂): | 70.00 | wt % |
| SAMPLE A-2: | | |
| Potassium superoxide (KO₂): | 35.00 | wt % |
| Calcium hydroxide (Ca(OH)₂): | 60.00 | wt % |
| Hopcalite: | 2.00 | wt % |
| Sodium silicate: | 3.00 | wt % |
| SAMPLE A-3: | | |
| Potassium superoxide (KO₂): | 35.00 | wt % |
| Aluminum hydroxide (Al(OH)₃): | 60.00 | wt % |
| Hopcalite: | 2.00 | wt % |
| Sodium silicate: | 3.00 | wt % |
| SAMPLE A-4: | | |
| Potassium superoxide (KO₂): | 35.00 | wt % |
| Magnesium hydroxide (Mg(OH)₂): | 60.00 | wt % |
| Hopcalite: | 2.00 | wt % |
| Sodium silicate: | 3.00 | wt % |
| SAMPLE A-5: | | |
| Potassium superoxide (KO₂): | 35.00 | wt % |
| Calcium hydroxide (Ca(OH)₂): | 60.00 | wt % |
| Hopcalite: | 2.00 | wt % |
| Glass fiber: | 3.00 | wt % |
| SAMPLE A-6: | | |
| Potassium superoxide (KO₂): | 35.00 | wt % |
| Calcium hydroxide (Ca(OH)₂): | 60.00 | wt % |
| Hopcalite: | 2.00 | wt % |
| Bentonite: | 3.00 | wt % |
| SAMPLE A-7: | | |
| Potassium superoxide (KO₂): | 35.00 | wt % |
| Calcium hydroxide (Ca(OH)₂): | 60.00 | wt % |
| Hopcalite: | 2.00 | wt % |
| Kaolinite: | 3.00 | wt % |
| SAMPLE B-1: | | |
| Sodium peroxide (Na₂O₂): | 30.00 | wt % |
| Calcium hydroxide (Ca(OH)₂): | 70.00 | wt % |
| SAMPLE B-2: | | |
| Sodium peroxide (Na₂O₂): | 35.00 | wt % |
| Calcium hydroxide (Ca(OH)₂): | 60.00 | wt % |
| Hopcalite: | 2.00 | wt % |
| Sodium silicate: | 3.00 | wt % |
| SAMPLE B-3: | | |
| Sodium peroxide (Na₂O₂): | 35.00 | wt % |
| Aluminum hydroxide (Al(OH)₃): | 60.00 | wt % |
| Hopcalite: | 2.00 | wt % |
| Sodium silicate: | 3.00 | wt % |
| SAMPLE B-4: | | |
| Sodium peroxide (Na₂O₂): | 35.00 | wt % |
| Magnesium hydroxide (Mg(OH)₂): | 60.00 | wt % |
| Hopcalite: | 2.00 | wt % |
| Sodium silicate: | 3.00 | wt % |
| SAMPLE B-5: | | |
| Sodium peroxide (Na₂O₂): | 35.00 | wt % |
| Calcium hydroxide (Ca(OH)₂): | 60.00 | wt % |
| Hopcalite: | 2.00 | wt % |
| Glass fiber: | 3.00 | wt % |
| SAMPLE B-6: | | |
| Sodium peroxide (Na₂O₂): | 35.00 | wt % |
| Calcium hydroxide (Ca(OH)₂): | 60.00 | wt % |
| Hopcalite: | 2.00 | wt % |
| Bentonite: | 3.00 | wt % |
| SAMPLE B-7: | | |
| Sodium peroxide (Na₂O₂): | 35.00 | wt % |
| Calcium hydroxide (Ca(OH)₂): | 60.00 | wt % |
| Hopcalite: | 2.00 | wt % |
| Kaolinite: | 3.00 | wt % |

### EXAMPLE 2

In this example, to test the reactivity of the oxygen generating compositions described in Example 1, 10 g of each of the compositions and 5 g of cotton were charged into a glass reactor, and then the glass reactor was heated at a rate of 1° C./min. to measure the ignition temperature.

The initial temperature of the reactor was 25° C. Each composition was tested ten times. The average ignition temperatures were calculated, and the results are listed in Tables 1 and 2 below.

TABLE 1
Reactivity of oxygen generating compositions based on potassium
superoxide

| Sample No. | Ignition temp.(° C.) | Material for stabilization of reactivity |
|---|---|---|
| A-1 | 245 | Ca(OH)₂ 70 wt % |
| A-2 | 231 | Ca(OH)₂ 60 wt % |
| A-3 | 325 | Al(OH)₃ 60 wt % |
| A-4 | 278 | Mg(OH)₂ 60 wt % |
| A-5 | 228 | Ca(OH)₂ 60 wt % |
| A-6 | 245 | Ca(OH)₂ 60 wt % |
| A-7 | 262 | Ca(OH)₂ 60 wt % |
| Pure KO₂ | 28 | — |

TABLE 2
Reactivity of oxygen generating
compositions based on sodium peroxide

| Sample No. | Ignition temp.(° C.) | Material for stabilization of reactivity |
|---|---|---|
| B-1 | 277 | Ca(OH)₂ 70 wt % |
| B-2 | 265 | Ca(OH)₂ 60 wt % |
| B-3 | 355 | Al(OH)₃ 60 wt % |
| B-4 | 305 | Mg(OH)₂ 60 wt % |
| B-5 | 253 | Ca(OH)₂ 60 wt % |
| B-6 | 275 | Ca(OH)₂ 60 wt % |
| B-7 | 277 | Ca(OH)₂ 60 wt % |
| Pure Na₂O₂ | 31 | — |

As can be seen from the ignition experimental results shown in Tables 1 and 2, pure potassium superoxide was ignited at a temperature (28° C.) close to room temperature, and pure sodium peroxide was ignited at 31° C. In contrast, the samples stabilized in accordance with the present invention were ignited at temperatures exceeding 200° C., which indicates that the oxidizing power of potassium superoxide and sodium peroxide is highly stabilized.

### EXAMPLE 3

In this example, to test the processability of the oxygen generating compositions, the compressive strength of the oxygen generating compositions in the shape of pellets prepared in Example 1 were measured.

TABLE 3
Compressive strength of oxygen
generating compositions according to the
kind of binders

| | Compressive | |
|---|---|---|
| Sample No. | strength (kg_{f}/cm²) | Binder |
| A-1 | 2.3 | — |
| A-2 | 11.2 | Sodium silicate 3.00 wt % |
| A-3 | 12.4 | Sodium silicate 3.00 wt % |
| A-4 | 10.7 | Sodium silicate 3.00 wt % |
| A-5 | 13.1 | Glass fiber 3.00 wt % |
| A-6 | 12.8 | Bentonite 3.00 wt % |
| A-7 | 11.7 | Kaolinite 3.00 wt % |
| B-1 | 2.6 | — |
| B-2 | 10.4 | Sodium silicate 3.00 wt % |
| B-3 | 9.4 | Sodium silicate 3.00 wt % |
| B-4 | 9.3 | Sodium silicate 3.00 wt % |
| B-5 | 11.2 | Glass fiber 3.00 wt % |
| B-6 | 12.5 | Bentonite 3.00 wt % |
| B-7 | 12.3 | Kaolinite 3.00 wt % |
| Pure KO₂ | 1.1 | — |
| Pure Na₂O₂ | 1.3 | — |

Referring to the experimental results shown in Table 3, the compressive strength of the oxygen generating compositions containing the respective binders was higher than that of the oxygen generating compositions containing no binder.

These results show that the binder-containing compositions are more rigid than pure potassium superoxide and sodium peroxide and can thus be molded into various shapes.

### EXAMPLE 4

100 g of each of the oxygen generating compositions described in Example 1 was placed in a 2L flask, and then nitrogen containing 5,000 ppm carbon dioxide (CO₂) was charged into the flask. The change in the concentration of carbon dioxide was recorded as a function of time.

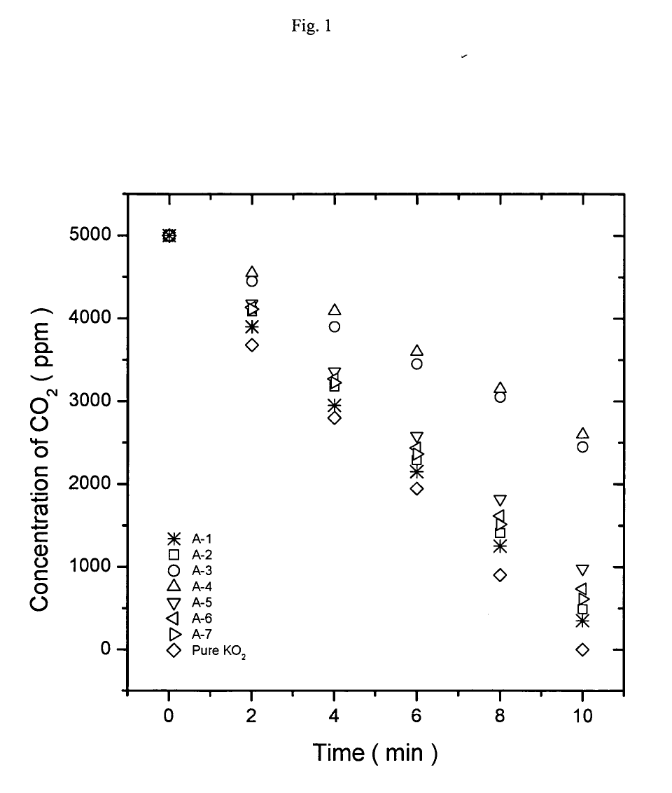

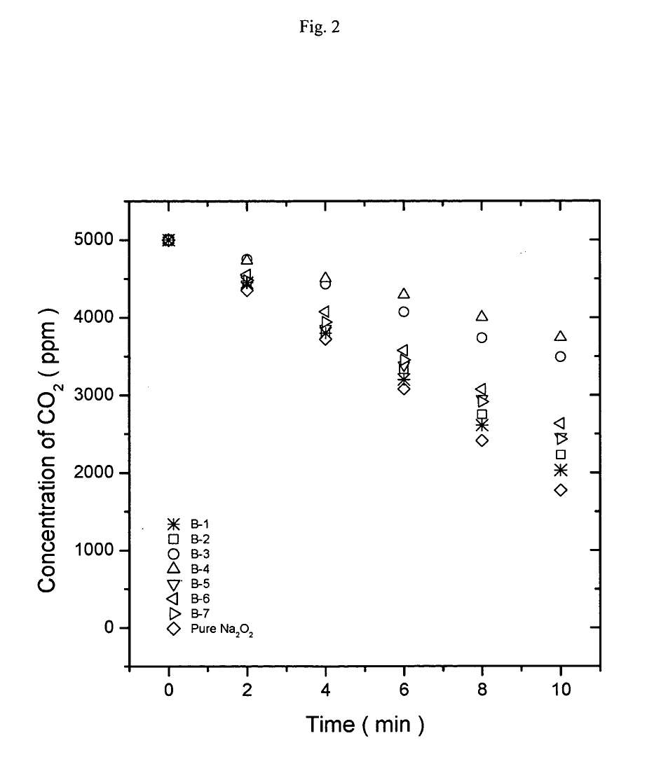

FIG. 1 is a graph showing the results of carbon dioxide absorption of the oxygen generating compositions based on potassium superoxide. Referring to FIG. 1, the composition (A-3) containing aluminum hydroxide and the composition (A-4) containing magnesium hydroxide showed much slower carbon dioxide absorption rate than the composition containing calcium hydroxide. FIG. 2 is a graph showing the results of carbon dioxide absorption of the oxygen generating compositions based on sodium peroxide. Referring to FIG. 2, among the oxygen generating compositions based on sodium peroxide, the compositions containing aluminum hydroxide or magnesium hydroxide showed much slower carbon dioxide absorption rate than the composition containing calcium hydroxide.

### EXAMPLE 5

1,000 g of each of the oxygen generating compositions described in Example 1 was placed in a 2L flask, and then nitrogen containing 5,000 ppm carbon monoxide was charged into the flask. The change in the concentration of carbon monoxide was recorded as a function of time.

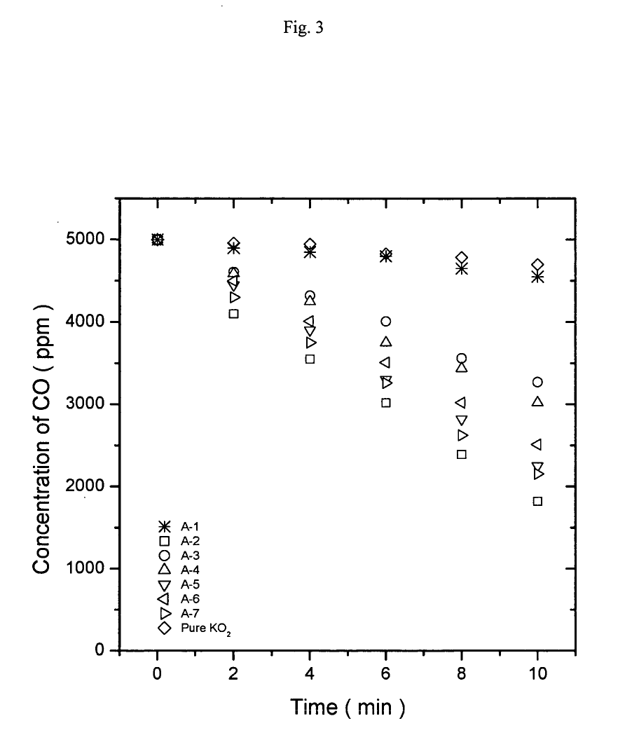

FIG. 3 is a graph showing the results of carbon monoxide absorption of the oxygen generating compositions comprising potassium superoxide. Referring to FIG. 3, pure potassium superoxide containing no hopcalite, and the oxygen generating composition comprising potassium superoxide and calcium hydroxide only showed very slow carbon monoxide absorption rates.

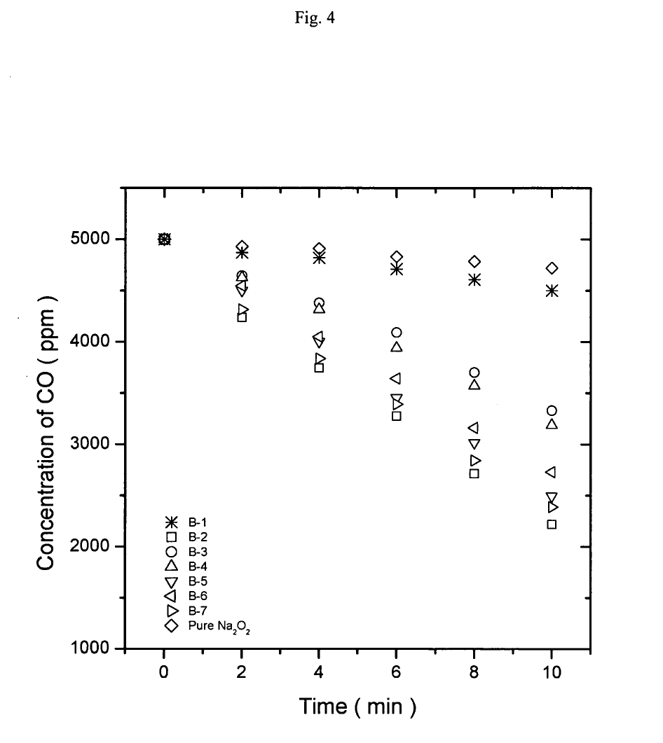

FIG. 4 is a graph showing the results of carbon monoxide absorption of the oxygen generating compositions comprising sodium peroxide. Referring to FIG. 4, similarly to the oxygen generating compositions comprising potassium superoxide, the oxygen generating compositions containing no hopcalite showed very slow carbon monoxide absorption rate.

### EXAMPLE 6

Oxygen generating compositions comprising sodium hydroxide were prepared in the same procedure as in Example 1.

| SAMPLE C-1 | | |
|---|---|---|
| Potassium superoxide (KO₂): | 35.00 | wt % |
| Calcium hydroxide (Ca(OH)₂): | 55.00 | wt % |
| Hopcalite: | 2.00 | wt % |
| Sodium silicate: | 3.00 | wt % |
| Sodium hydroxide: | 5.00 | wt % |
| SAMPLE C-2 | | |
| Sodium peroxide (Na₂O₂): | 35.00 | wt % |
| Calcium hydroxide (Ca(OH)₂): | 55.00 | wt % |
| Hopcalite: | 2.00 | wt % |
| Sodium silicate: | 3.00 | wt % |
| Sodium hydroxide: | 5.00 | wt % |

### EXAMPLE 7

1,000 g of each of the oxygen generating compositions (SAMPLE Nos. C-1 and C-2) described in Example 6, 1,000 g of pure potassium superoxide and 1,000 g of pure sodium peroxide were placed in 2L flasks, respectively. Thereafter, nitrogen containing 5,000 ppm carbon dioxide (CO₂) was charged into the flasks. The change in the concentration of carbon dioxide was recorded as a function of time.

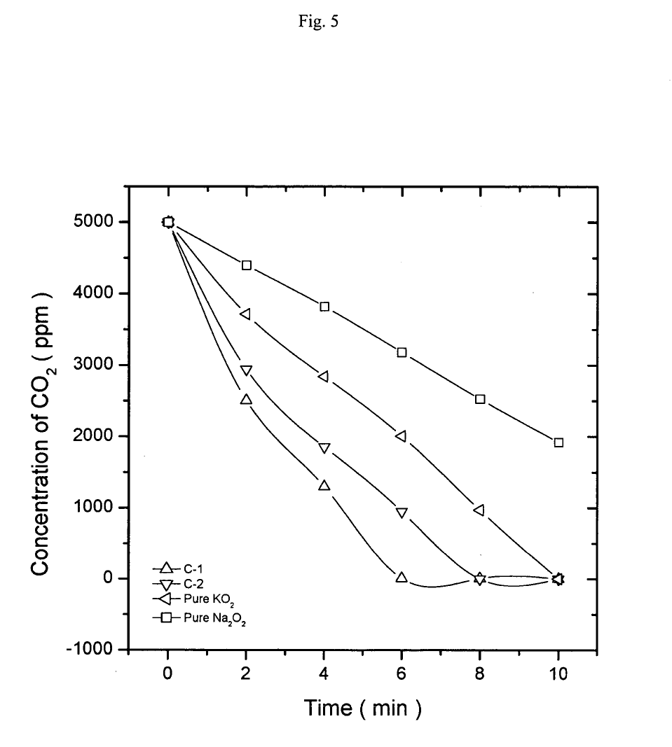

The results are shown in FIG. 5. The oxygen generating compositions containing sodium hydroxide showed very fast carbon dioxide absorption rate, compared to pure potassium superoxide and pure sodium peroxide. This indicates that the addition of sodium hydroxide increases initial reaction rate of composition, i.e., the initial carbon dioxide absorption rate.

### INDUSTRIAL APPLICABILITY

As apparent from the above description, since the oxygen generating compositions according to the present invention function to absorb carbon dioxide, carbon monoxide, SOₓ and NOₓ and transform them into oxygen, they can be widely used as a various air purifier.

In particular, since the oxygen generating compositions according to the present invention have a very high compressive strength compared to pure potassium superoxide, they can be manufactured into a flat-plate filter capable of being mounted on apparatuses, such as air conditioners and air cleaners. Therefore, the oxygen generating compositions according to the present invention are industrially applicable.

The foregoing embodiments do not serve to limit the present invention. It should be understood that various modifications and changes can be made without departing from the scope and spirit of the present invention as disclosed in the appended claims.

1. An oxygen generating composition comprising 20˜90 wt % of potassium superoxide and 10˜80 wt % of a material for stabilizing the reactivity and oxidizing power of the potassium superoxide.

2. An oxygen generating composition comprising 20˜90 wt % of sodium peroxide and 10˜80 wt % of a material for stabilizing the reactivity and oxidizing power of the sodium peroxide.

3. The oxygen generating composition according to claim 1, wherein the material for stabilizing the reactivity and oxidizing power is at least one material selected from the group consisting of calcium hydroxide (Ca(OH)₂), aluminum hydroxide (Al(OH)₃), magnesium hydroxide (Mg(OH)₂), barium hydroxide (Ba(OH)₂), calcium carbonate (CaCO₃), talc and clay.

4. An oxygen generating composition comprising 95˜99.99 wt % of the oxygen generating composition according to claim 1 and 0.01˜5 wt % of an oxidation catalyst of carbon monoxide.

5. The oxygen generating composition according to claim 4, wherein the oxidation catalyst of carbon monoxide is at least one compound selected from the group consisting of copper oxide (CuO), manganese oxide (MnO) and a mixture thereof (hopcalite).

6. An oxygen generating composition comprising 90˜99.99 wt % of the oxygen generating composition according to claim 1 and 0.01˜10 wt % of a material for improving the moldability and processability of the composition.

7. The oxygen generating composition according to claim 6, wherein the material for improving the moldability and processability is at least one species selected from the group consisting of glass powder, glass fiber, ceramic fiber, steel wool, bentonite, kaolinite, sodium silicate and potassium silicate.

8. An oxygen generating composition comprising 90˜99.99 wt % of the oxygen generating composition according to claim 1 and 0.01˜10 wt % of a base for increasing initial carbon dioxide absorption rate.

9. The oxygen generating composition according to claim 8, wherein the base is at least one base selected from the group consisting of sodium hydroxide, lithium hydroxide and potassium hydroxide.

10. An oxygen generating composition comprising 85˜99.98 wt % of the oxygen generating composition according to claim 1, 0.01˜5 wt % of an oxidation catalyst of carbon monoxide and 0.01˜10 wt % of a material for improving the moldability and processability of the composition.

11. An oxygen generating composition comprising 80˜99.98 wt % of the oxygen generating composition according to claim 1, 0.01˜10 wt % of a material for improving the moldability and processability of the composition, and 0.01˜10 wt % of a base for increasing Initial carbon dioxide absorption rate.

12. An oxygen generating composition comprising 85˜99.98 wt % of the oxygen generating composition according to claim 1, 0.01˜5 wt % of an oxidation catalyst of carbon monoxide, and 0.01˜10 wt % of a base for increasing initial carbon dioxide absorption rate.

13. An oxygen generating composition comprising 75˜99.97 wt % of the oxygen generating composition according to claim 1, 0.01˜5 wt % of an oxidation catalyst of carbon monoxide, 0.01˜10 wt % of a material for improving the moldability and processability of the composition, and 0.01˜10 wt % pf a base for increasing initial carbon dioxide absorption rate.

14. The oxygen generating composition according to claim 2, wherein the material for stabilizing the reactivity and oxidizing power is at least one material selected from the group consisting of calcium hydroxide (Ca(OH)₂), aluminum hydroxide (Al(OH)₃), magnesium hydroxide (Mg(OH)₂), barium hydroxide (Ba(OH)₂), calcium carbonate (CaCO₃), talc and clay.

15. An oxygen generating composition comprising 95˜99.99 wt % of the oxygen generating composition according to claim 2 and 0.01˜5 wt % of an oxidation catalyst of carbon monoxide.

16. The oxygen generating composition according to claim 15, wherein the oxidation catalyst of carbon monoxide is at least one compound selected from the group consisting of copper oxide (CuO), manganese oxide (MnO) and a mixture thereof (hopcalite).

17. An oxygen generating composition comprising 90˜99.99 wt % of the oxygen generating composition according to claim 2 and 0.01˜10 wt % of a material for improving the moldability and processability of the composition.

18. The oxygen generating composition according to claim 17, wherein the material for improving the moldability and processability is at least one species selected from the group consisting of glass powder, glass fiber, ceramic fiber, steel wool, bentonite, kaolinite, sodium silicate and potassium silicate.

19. An oxygen generating composition comprising 90˜99.99 wt % of the oxygen generating composition according to claim 2 and 0.01˜10 wt % of a base for increasing initial carbon dioxide absorption rate.

20. The oxygen generating composition according to claim 19, wherein the base is at least one base selected from the group consisting of sodium hydroxide, lithium hydroxide and potassium hydroxide.

21. An oxygen generating composition comprising 85˜99.98 wt % of the oxygen generating composition according to claim 2, 0.01˜5 wt % of an oxidation catalyst of carbon monoxide and 0.01˜10 wt % of a material for improving the moldability and processability of the composition.

22. An oxygen generating composition comprising 80˜99.98 wt % of the oxygen generating composition according to claim 2, 0.01˜10 wt % of a material for improving the moldability and processability of the composition, and 0.01˜10 wt % of a base for increasing initial carbon dioxide absorption rate.

23. An oxygen generating composition comprising 85˜99.98 wt % of the oxygen generating composition according to claim 2, 0.01˜5 wt % of an oxidation catalyst of carbon monoxide, and 0.01˜10 wt % of a base for increasing initial carbon dioxide absorption rate.

24. An oxygen generating composition comprising 75˜99.97 wt % of the oxygen generating composition according to claim 2, 0.01˜5 wt % of an oxidation catalyst of carbon monoxide, 0.01˜10 wt % of a material for improving the moldability and processability of the composition, and 0.01˜10 wt % pf a base for increasing initial carbon dioxide absorption rate.
